(19) United States
(12) Patent Application Publication
(10) Pub. No.: US 2023/0372268 A1
(43) Pub. Date: Nov. 23, 2023

(54) METHODS OF INTRAVENOUSLY ADMINISTERING SOTALOL

(71) Applicant: AltaThera Pharmaceuticals LLC, Chicago, IL (US)

(72) Inventor: Brandon Ira Kashfian, Chicago, IL (US)

(21) Appl. No.: 18/324,703

(22) Filed: May 26, 2023

Related U.S. Application Data
(63) Continuation-in-part of application No. 17/585,190, filed on Jan. 26, 2022.
Application No. 17/585,190 is a continuation of application No. 16/863,567, filed on Apr. 30, 2020, now abandoned.
Application No. 16/863,567 is a continuation-in-part of application No. 16/693,312, filed on Nov. 24, 2019.
Application No. 16/693,312 is a continuation of application No. 16/103,815, filed on Aug. 14, 2018, now Pat. No. 10,512,620.
Application No. 16/863,567 is a continuation-in-part of application No. 16/693,310, filed on Nov. 24, 2019, now Pat. No. 11,696,902.
Application No. 16/693,310 is a continuation-in-part of application No. 16/103,815, filed on Aug. 14, 2018, now Pat. No. 10,512,620.
Application No. 16/103,815 is a continuation-in-part of application No. 17/306,490, filed on May 3, 2021.
Application No. 17/306,490 is a continuation of application No. 16/849,099, filed on Apr. 15, 2020, now abandoned.
Application No. 17/306,490 is a continuation-in-part of application No. 16/693,312, filed on Nov. 24, 2019.
Application No. 16/693,312 is a continuation-in-part of application No. 16/693,310, filed on Nov. 24, 2019, now Pat. No. 11,696,902.
(60) Provisional application No. 63/346,003, filed on May 26, 2022.
(60) Provisional application No. 63/009,511, filed on Apr. 14, 2020.
(60) Provisional application No. 62/987,832, filed on Mar. 10, 2020.

(51) Int. Cl. A61K 31/18; A61K 9/00; A61P 9/06

(52) U.S. Cl. CPC A61K 31/18; A61K 9/0019; A61P 9/06

(57) ABSTRACT

Methods of administering a 5-hour IV loading dose of sotalol and one or more oral maintenance dose(s) or one or more 5-hour IV maintenance dose(s) in amounts effective for treating a cardiovascular condition are described. Methods of administering a 1-hour IV loading dose of sotalol and one or more 5-hour IV maintenance dose(s) in amounts effective for treating a cardiovascular condition are also described. The subject can be discharged prior to administration of any maintenance doses, after administration of a single maintenance dose, or after administration of two or more maintenance doses.

### CROSS-REFERENCE TO RELATED APPLICATIONS

The present application claims priority to and the benefit of the filing date of U.S. Provisional Application No. 63/346,003, filed May 26, 2022. The present application is also a Continuation-in-Part (CIP) application of U.S. application Ser. No. 17/585,190 filed Jan. 26, 2022, which is a Continuation application of U.S. application Ser. No. 16/863,567, filed Apr. 30, 2020, which application claims priority to and the benefit of the filing date of U.S. Provisional Application No. 63/009,511, filed Apr. 14, 2020. The '567 application is a CIP application of U.S. application Ser. No. 16/693,312, filed Nov. 24, 2019, which application is a Continuation application of U.S. application Ser. No. 16/103,815, filed Aug. 14, 2018, which issued as U.S. Pat. No. 10,512,620 on Dec. 24, 2019. The '567 application is a CIP application of U.S. application Ser. No. 16/693,310, filed Nov. 24, 2019, which application is a CIP application of the '815 application. The present application is also a CIP application of U.S. application Ser. No. 17/306,490 filed May 3, 2021, which is a Continuation application of U.S. application Ser. No. 16/849,099, filed Apr. 15, 2020, which application claims priority to and the benefit of the filing date of U.S. Provisional Application No. 62/987,832, filed Mar. 10, 2020. The '490 application is also a CIP application of U.S. application Ser. No. 16/693,312 filed Nov. 24, 2019, which is a Continuation application of U.S. application Ser. No. 16/103,815 filed Aug. 14, 2018. The '490 application is a CIP application of U.S. application Ser. No. 16/693,310 filed Nov. 24, 2019, which is a CIP application of U.S. application Ser. No. 16/103,815 filed Aug. 14, 2018. The disclosures of each of these applications are hereby incorporated by reference herein in their entireties.

### BACKGROUND OF THE INVENTION

### Field of the Invention

The present invention relates to the field of cardiovascular pharmaceutics, more particularly compositions and methods for the treatment of cardiovascular conditions, such as arrhythmias, with intravenous anti-arrhythmics, such as sotalol hydrochloride.

### Description of Related Art

Sotalol hydrochloride is a racemic mixture of d- and 1-sotalol hydrochloride. Sotalol hydrochloride is a white, crystalline solid with a molecular weight of 308.8. It is hydrophilic, soluble in water, propylene glycol, and ethanol. Chemically, sotalol hydrochloride is a d,l-N-[4-[1-hydroxy-2-[(1-methylethyl)amino] ethyl] phenyl] methane-sulfonamide monohydrochloride. (HIGHLIGHTS OF PRESCRIBING INFORMATION: Sotalol hydrochloride injection for intravenous use, Revised July 2009). The molecular formula is C12H2ON2O3S·HCl and is represented by the following structural formula:

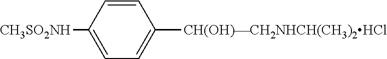

Intravenous sotalol is supplied as a sterile, clear solution in 10 ml vials, each vial containing 150 mg sotalol hydrochloride in acetate buffer. Sotalol hydrochloride is an antiarrhythmic drug with both beta adrenoreceptor blocking (Class II) and cardiac action potential duration prolongation (Class III) properties. The drug is hemodynamically well-tolerated and has a small risk of proarrhythmia. Both isomers have similar Class III activity, while the 1-isomer is responsible for virtually all of the beta blocking activity. Sotalol does not have partial beta agonist or membrane stabilizing activity (Na channel inhibition). Sotalol does not undergo metabolism and is nearly 100% bioavailable when taken on an empty stomach. Sotalol hydrochloride does not bind to plasma proteins. The pharmacokinetics of d- and 1-sotalol enantiomers are essentially identical. Excretion is predominantly via the kidney in the unchanged form and therefore lower doses are necessary in patients with renal impairment.

Sotalol is FDA approved for the maintenance of normal sinus rhythm in patients with history of highly symptomatic atrial fibrillation/flutter and for the treatment of documented life-threatening ventricular arrhythmias. Sotalol is currently approved in the US for oral administration (for example, under the brand name BETAPACE AF®, Bayer HealthCare Pharmaceuticals Inc.) and is approved for IV administration (AltaThera Pharmaceuticals LLC). Sotalol is an effective antiarrhythmic agent but also can cause serious ventricular arrhythmias, primarily Torsade de Pointes (TdP) type ventricular tachycardia, a polymorphic ventricular tachycardia associated with QTc prolongation. QTc prolongation is directly related to the plasma concentration of sotalol. Conventionally, steady-state plasma levels of sotalol and maximum QTc prolongation are obtained in 3 days with oral administration (i.e. after 5-6 doses when administered twice daily). QTc changes are directly related to maximum serum concentration of the sotalol. To minimize the risk of sotalol caused arrhythmias, the product label mandates that patients initiated or re-initiated on sotalol should be hospitalized for at least three days or until steady state drug levels are achieved, in a facility that can provide cardiac resuscitation and continuous electrocardiographic monitoring.

Hospitalization represents a high-cost burden in the United States. The three-day hospital stay is also burdensome to the patient, resulting in loss of productivity, time away from family, and an increased risk of hospital-acquired infections, as well as a diversion of hospital resources, staff, and patient care beds. The availability of IV sotalol offers an opportunity to improve on the sotalol loading protocol and decreases the length of hospital stay.

### SUMMARY OF THE INVENTION

Sotalol is a commonly used oral anti-arrhythmic drug for management of atrial arrhythmias. Due to a risk of pro-arrhythmia, inpatient initiation of sotalol with QTc monitoring for five doses (a typical three-day protocol) is a standard treatment. Patent literature relating to sotalol includes: U.S. Pat. Nos. 10,512,620, 10,799,138, 11,610,660, 11,583,216, and 11,344,518; and US Published Patent Application Nos. 2020/0093759, 2020/0085771, 2021/0283049, 2020/0253903, 2020/0338027, 2019/0307343, 2019/0380605, 2021/0076959, 2022/0142954, 2022/0241225, 2022/0339130, and 2023/0075398, which are incorporated by reference herein in their entireties. This disclosure provides a change from the guidelines for sotalol in-hospital loading that achieves steady-state blood level and maximum QTc prolongation within hours. Pharmacokinetic/pharmacodynamics (PK/PD) simulations indicate that administering an intravenous loading dose of sotalol can achieve a Cₘₐₓ at steady state and thus maximum QTc prolongation within hours. Thus, the present methods can enable physicians to evaluate the major safety concern, excessive QTc prolongation, in hours instead of days.

Aspects of the invention include Aspect 1, which is a method of intravenously administering sotalol, comprising: administering one or more IV dosage of sotalol to a subject, over a period of about 1-5 hours, and comprising about 20-260 mg sotalol, wherein in some embodiments at least one of the IV dosage(s) is an IV loading dose.

Aspect 2 is the method of Aspect 1, comprising administering a 1-hour IV loading dose of sotalol (with the amount of the dose based on creatinine clearance) and one or more 5-hour IV maintenance dose(s) in amounts effective for treating a cardiovascular condition.

Aspect 3 is the method of Aspect 1 or 2, comprising administering a 5-hour IV loading dose of sotalol and one or more 5-hour IV maintenance dose(s) in amounts effective for treating a cardiovascular condition.

Aspect 4 is a method of administering sotalol, comprising: administering a 1-hour IV loading dosage of sotalol hydrochloride to a subject, wherein an amount of the sotalol hydrochloride in the IV loading dose is based on a creatinine clearance of the subject; and following the IV loading dose, administering to the subject one or more 5-hour IV maintenance dose; wherein the IV loading and IV maintenance doses are administered as follows: the 1-hr IV loading dose comprises 60-125 mg of sotalol hydrochloride; and the 5-hr IV maintenance dose comprises 75-150 mg sotalol hydrochloride.

Aspect 5 is the method of any of Aspects 1-4, wherein the subject is being initiated on a 75 mg 5-hour IV maintenance dose and one of the following protocols (a)-(c) is selected: (a) the subject has a creatinine clearance of >90 mL/min; and the 1-hr IV loading dose comprises 60 mg of sotalol hydrochloride; (b) the subject has a creatinine clearance of 60-90 mL/min; and the 1-hr IV loading dose comprises 82.5 mg of sotalol hydrochloride; or (c) the subject has a creatinine clearance of 30-60 mL/min or 10-30 mL/min; and the 1-hr IV loading dose comprises 75 mg of sotalol hydrochloride.

Aspect 6 is the method of any of Aspects 1-5, wherein the subject is being initiated on a 112.5 mg 5-hour IV maintenance dose and one of the following protocols (a)-(c) is selected: (a) the subject has a creatinine clearance of >90 mL/min; and the 1-hr IV loading dose comprises 90 mg of sotalol hydrochloride; (b) the subject has a creatinine clearance of 60-90 mL/min; and the 1-hr IV loading dose comprises 125 mg of sotalol hydrochloride; or (c) the subject has a creatinine clearance of 30-60 mL/min or 10-30 mL/min; and the 1-hr IV loading dose comprises 112.5 mg of sotalol hydrochloride.

Aspect 7 is the method of any of Aspects 1-6, wherein the subject is being escalated from an 80 mg oral maintenance dose to a 112.5 mg 5-hour IV maintenance dose or is being escalated from an 75 mg IV maintenance dose to a 112.5 mg 5-hour IV maintenance dose and one of the following protocols (a)-(b) is selected: (a) the subject has a creatinine clearance of >90 mL/min; and the 1-hr IV loading dose comprises 75 mg of sotalol hydrochloride; (b) the subject has a creatinine clearance of 10-90 mL/min; and the 1-hr IV loading dose comprises 82.5 mg of sotalol hydrochloride.

Aspect 8 is the method of any of Aspects 1-7, wherein the subject is being escalated from a 120 mg oral maintenance dose to a 150 mg 5-hour IV maintenance dose or is being escalated from a 112.5 mg IV maintenance dose to a 150 mg 5-hour IV maintenance dose and one of the following protocols (a)-(b) is selected: (a) the subject has a creatinine clearance of >90 mL/min; and the 1-hr IV loading dose comprises 90 mg of sotalol hydrochloride; (b) the subject has a creatinine clearance of 10-90 mL/min; and the 1-hr IV loading dose comprises 105 mg of sotalol hydrochloride.

Aspect 9 is the method of any of Aspects 1-6, wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension.

Aspect 10 is a method of administering sotalol, comprising: administering a 5-hour IV loading dosage of sotalol hydrochloride to a subject; following the IV loading dose, administering to the subject one or more 5-hour IV maintenance dose or one or more oral maintenance dose.

Aspect 11 is the method of Aspect 10, wherein: the subject is being initiated on a 75 mg IV maintenance dose and the subject is administered 50-100 mg for the IV loading dose.

Aspect 12 is the method of Aspect 10, wherein: the subject is being initiated on a 112.5 mg IV maintenance dose and the subject is administered 75-130 mg for the IV loading dose.

Aspect 13 is the method of Aspect 10, wherein: the subject is being escalated from a 75 mg IV maintenance dose to a 112.5 mg IV maintenance dose; and the subject is administered 60-115 mg for the IV loading dose.

Aspect 14 is the method of Aspect 10, wherein: the subject is being escalated from a 112.5 mg IV maintenance dose to a 150 mg IV maintenance dose; and the subject is administered 70-150 mg for the IV loading dose.

Aspect 15 is the method of Aspect 10, wherein: the subject is being escalated from a 150 mg IV maintenance dose to a 225 mg IV maintenance dose; and the subject is administered 100-225 mg for the IV loading dose.

Aspect 16 is the method of Aspect 10, wherein: the subject is being initiated on an 80 mg oral maintenance dose and the subject is administered 50-100 mg for the IV loading dose.

Aspect 17 is the method of Aspect 10, wherein: the subject is being initiated on a 120 mg oral maintenance dose and the subject is administered 75-130 mg for the IV loading dose.

Aspect 18 is the method of Aspect 10, wherein: the subject is being escalated from an 80 mg oral maintenance dose to a 120 mg oral maintenance dose; and the subject is administered 60-115 mg for the IV loading dose.

Aspect 19 is the method of Aspect 10, wherein: the subject is being escalated from a 120 mg oral maintenance dose to a 160 mg oral maintenance dose; and the subject is administered 70-150 mg for the IV loading dose.

Aspect 20 is the method of Aspect 10, wherein: the subject is being escalated from a 160 mg oral maintenance dose to a 240 mg oral maintenance dose; and the subject is administered 100-225 mg for the IV loading dose.

Aspect 21 is the method of Aspect 10, wherein: wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension.

Aspect 22 is a method of administering sotalol, comprising: (A) before administering a 1-hour IV loading dosage of sotalol to a subject, determining a creatinine clearance (CrCl) of the subject, and selecting an amount of sotalol to administer to the subject based on the creatinine clearance; (B) administering to the subject the 1-hour IV loading dosage comprising the selected amount of sotalol based on the CrCl, wherein the 1-hour IV loading dosage is administered to the subject in a facility capable of providing cardiac resuscitation and continuous electrocardiographic monitoring; (C) administering to the subject a 5-hour IV maintenance dose of sotalol at a selected period of time from the IV loading dose; and (D) administering one or more subsequent IV maintenance doses of sotalol to the subject at a second selected period of time from the first IV maintenance dose and at a selected interval therefrom.

Aspect 23 is the method of Aspect 22, wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension.

Aspect 24 is the method of Aspect 23, further comprising: obtaining a QT or QTc interval of the subject before administration of the 1-hour IV loading dosage; and obtaining a QT or QTc interval of the subject during or after administration of the 1-hour IV loading dosage, but before administration of one or more 5-hour IV maintenance dose; wherein when the QT or QTc interval during or after administering the 1-hour IV loading dosage is less than a 20% increase from the QT or QTc interval before administering the 1-hour IV loading dosage, then administering one or more 5-hour IV maintenance dose of sotalol hydrochloride.

Aspect 25 is the method of Aspect 22, further comprising: obtaining a QT or QTc interval after administering a first 5-hour IV maintenance dose; when the QT or QTc interval obtained after administering the first 5-hour IV maintenance dose is equal to or more than a 20% increase from the QT or QTc interval before administering the 1-hour IV loading dosage, then administering a second 5-hour IV maintenance dose comprising an amount of sotalol hydrochloride that is less than the amount of sotalol hydrochloride as the first 5-hour IV maintenance dose.

Aspects of the invention include Aspect 1A, which is a method of intravenously administering sotalol, comprising: administering an IV loading dosage of sotalol to a subject over a period of about 1-5 hours, such as over 1 hour, 2 hours, 3 hours, 4 hours or 5 hours, such as in the range of 49.5 mg to 140.8 mg; and wherein the IV loading dosage and period are selected to enable maximum serum concentration at steady state of the subject within 24 hours of the start of administering the IV loading dose, such as within 1 hour. In embodiments, the Cₘₐₓ at steady state for the subject can be within a range of ±25% of a Cₘₐₓ at steady state predicted for the subject for an oral dosing protocol of 80 mg, 120 mg, or 160 mg or 240 mg.

Aspect 2A is the method of Aspect 1A, comprising: administering the IV loading dosage in an amount of 20-141 mg; and after administering the IV loading dosage, administering one or more oral dosage of sotalol to the subject in an amount ranging from 80-240 mg.

Aspect 3A is the method of Aspect 1A or 2A, further comprising administering or repeating oral administration of sotalol at least once at a selected interval.

Aspect 4A is the method of any of Aspects 1A-3A, wherein: the IV loading dosage is administered while the subject is in a facility capable of providing cardiac resuscitation and electrocardiographic monitoring; and oral administration of one or more oral dosages of sotalol is performed before or after the subject is discharged from the facility capable of providing cardiac resuscitation and electrocardiographic monitoring.

Aspect 5A is the method of any of Aspects 1A-4A, wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension.

Aspect 6A is the method of any of Aspects 1A-5A, wherein the subject is currently in sinus rhythm and/or has experienced cardioversion to sinus rhythm.

Aspect 7A is the method of any of Aspects 1A-6A, wherein: the IV loading dosage is administered in an amount of: about 55-128 mg, about 73.8-105.6 mg, or about 88-141 mg.

Aspect 8A is the method of any of Aspects 1A-7A, wherein: the oral dosing protocol comprises one or more oral dose in an amount of 80 mg, 120 mg, 160 mg or 240 mg.

Aspect 9A is the method of any of Aspects 1A-8A, wherein the IV loading dosage is administered in an amount of: (a) about 55-128 mg, for the subject who is naïve to sotalol; (b) about 73.8-105.6 mg, for the subject who had received 80 mg of oral sotalol hydrochloride prior to the IV loading dosage; or (c) about 88-141 mg, for the subject who had received 120 mg of oral sotalol hydrochloride prior to the IV loading dosage.

Aspect 10A is the method of any of Aspects 1A-9A, wherein the oral dosage is administered to the subject after completion of the IV loading dosage.

Aspect 11A is the method of any of Aspects 1A-10A, wherein the IV loading dosage is administered as a single IV loading dosage or by way of several IV loading doses.

Aspect 12A is the method of any of Aspects 1A-11A, wherein: the IV loading dosage is administered in an amount of about 55-128 mg and the oral dosing protocol comprises one or more oral dose in an amount of 80-120 mg; or the IV loading dosage is administered in an amount of about 73.8-105.6 mg and the oral dosing protocol comprises one or more oral dose in an amount of 120-160 mg; or the IV loading dosage is administered in an amount of about 88-141 mg and the oral dosing protocol comprises one or more oral dose in an amount of 120-160 mg.

Aspect 13A is the method of any of Aspects 1A-12A, wherein: the subject is being initiated or escalated to a higher dose of oral sotalol; the administering of the IV loading dosage comprises: determining kidney function, such as a creatinine clearance, of the subject; determining a QTc of the subject; administering to the subject an IV loading dosage of sotalol, wherein the IV loading dosage is selected from an amount ranging from about 55-128 mg, and determining a second QTc of the subject.

Aspect 14A is the method of any of Aspects 1A-13A, wherein: if one or more oral dose(s) of sotalol are administered to the subject, determining one or more subsequent QTc of the subject between one or more of the oral doses; and administering one or more subsequent oral doses to the subject wherein the oral dose administered is selected from 80-240 mg.

Aspect 15A is the method of any of Aspects 1A-14A, wherein if one or more oral dose(s) of sotalol are administered to the subject, the administering of the oral dose(s) begins within about 12 hours after completion of the administering of the IV loading dose, such as beginning about immediately after, or up to 1 hour after, or up to 2 hours after, or about 2-4 hours after, or about 4-6 hours, 6-8 hours, 8-10 hours, or 10-12 hours after.

Aspect 16A is the method of any of Aspects 1A-15A, wherein if one or more oral dose(s) of sotalol are administered to the subject, the administering of the oral dose(s) occurs at a 12-hour, 24-hour or 48-hour interval from a previous oral dose.

Aspect 17A is the method of any of Aspects 1A-16A, wherein if one or more oral dose(s) of sotalol are administered to the subject, a second oral dose of sotalol is administered to the subject about 12-48 hours after a first oral dose.

Aspect 18A is the method of any of Aspects 1A-17A, wherein the IV loading dose is administered to the subject in an amount of about 55-105.6 mg and one or more maintenance dose(s) is administered to the subject in an amount of 160 mg.

Aspect 19A is the method of any of Aspects 1A-18A, wherein the subject had received a prior dose of sotalol prior to administration of the IV loading dose, such as about 12-24 hours prior to administration of the IV loading dose.

Aspect 20A is the method of any of Aspects 1A-19A, wherein the subject is capable of experiencing a sotalol Cₘₐₓ at steady state within 24 hours, such as within 1 hour, of the start of administering the IV loading dose and/or within a range of ±25% of a Cₘₐₓ at steady state predicted for the subject for an oral dosing protocol of 80 mg, 120 mg, 160 mg or 240 mg sotalol, such as about 75%, 80%, 85%, 87%, 90%, 92%, 95%, 97% or 99%.

Aspect 21A is the method of any of Aspects 1A-20A, wherein the subject has a creatinine clearance of >60 mL/min before being administered the IV loading dose of sotalol.

Aspect 22A is the method of any of Aspects 1A-20A, wherein the subject has a creatinine clearance of 40-60 mL/min before being administered the IV loading dose of sotalol.

Aspect 23A is the method of any of Aspects 1A-20A, wherein the subject has a creatinine clearance of 20 mL/min to <40 mL/min before the IV loading dose of sotalol.

Aspect 24A is the method of any of Aspects 1A-23A, wherein if one or more oral dose(s) of sotalol are administered to the subject, the oral dosing of sotalol is administered at a 12 hour or 48 hour interval, optionally wherein the subject is discharged with instructions to self-administer the oral dose and/or one or more subsequent oral doses.

Aspect 25A is the method of any of Aspects 1A-24A, further comprising measuring a QT interval or QTc of the subject before administration of the IV loading dose.

Aspect 26A is the method of any of Aspects 1A-25A, further comprising measuring a QT interval or QTc of the subject after administration of the IV loading dose and if an oral dose is administered, then before the first oral dose.

Aspect 27A is the method of any of Aspects 1A-26A, further comprising measuring a QT interval or QTc of the subject after administration of a second oral dose, if any, or any subsequent oral dose.

Aspect 28A is the method of any of Aspects 1A-27A, wherein the amount of the oral dose, if any, is a lower amount than the IV loading dosage, or the amount of the oral dose is a higher amount than the IV loading dosage.

Aspect 29A is the method of any of Aspects 1A-28A, wherein: the subject is identified as non-renally impaired by having a creatinine clearance in the range of 60-90 mL/min and the IV loading dosage is 60 mg; or the subject is identified as renally impaired as indicated by having a creatinine clearance in the range of 10-30 mL/min and the IV loading dosage is 75 mg; and after the administering of the IV loading dosage, administering an 80 mg oral dose of sotalol hydrochloride to the non-renally impaired subject or to the renally impaired subject.

Aspect 30A is the method of any of Aspects 1A-28A, wherein: the subject is identified as non-renally impaired by having a creatinine clearance in the range of 60-90 mL/min and the IV loading dosage is 90 mg; or the subject is identified as renally impaired as indicated by having a creatinine clearance in the range of 10-30 mL/min and the IV loading dosage is 112.5 mg; and after the administering of the IV loading dosage, administering a 120 mg oral dose of sotalol hydrochloride to the non-renally impaired subject or to the renally impaired subject.

Aspect 31A is the method of any of Aspects 1A-28A, wherein: the subject is identified as non-renally impaired by having a creatinine clearance in the range of 60-90 mL/min and the IV loading dosage is 75 mg; or the subject is identified as renally impaired as indicated by having a creatinine clearance in the range of 10-30 mL/min and the IV loading dosage is 82.5 mg; and after the administering of the IV loading dosage, administering a 120 mg oral dose of sotalol hydrochloride to the non-renally impaired subject or to the renally impaired subject.

Aspect 32A is the method of any of Aspects 1A-28A, wherein: the subject is identified as non-renally impaired by having a creatinine clearance in the range of 60-90 mL/min and the IV loading dosage is 90 mg; or the subject is identified as renally impaired as indicated by having a creatinine clearance in the range of 10-30 mL/min and the IV loading dosage is 105 mg; and after the administering of the IV loading dosage, administering a 160 mg oral dose of sotalol hydrochloride to the non-renally impaired subject or to the renally impaired subject.

Aspect 33A is the method of any of Aspects 1A-32A, wherein the subject is identified as having symptomatic atrial fibrillation and/or atrial flutter, and optionally is in sinus rhythm.

Aspect 34A is the method of any of Aspects 1A-33A, wherein the IV loading dose is administered to the subject in an amount and over a period of time such that the sotalol reaches or is capable of reaching a Cₘₐₓ at steady state in the subject that is at least about 75%, 80%, 85%, 87%, 90%, 92%, 95%, 97% or 99% of a Cₘₐₓ at steady state for an oral sotalol dosing protocol of 80 mg, or 120 mg, or 160 mg or 240 mg for the subject.

### DETAILED DESCRIPTION OF VARIOUS EMBODIMENTS OF THE INVENTION

Reference will now be made in detail to various illustrative implementations. It is to be understood that the following discussion of implementations is not intended to be limiting.
AF is atrial fibrillation.
AFL is atrial flutter.
AF/AFL is atrial fibrillation and/or atrial flutter.
IV is intravenous.
PO means “per os” and refers to an oral dosing regimen.
BID means “bis in die” and means twice a day.
QD means “quaque die” and means once a day.
QID means “quater in die” and means four times a day.
Patient (or subject) refers to a human patient.
BP is blood pressure.
HR is heart rate.

Renally impaired refers to patients having creatinine clearance rates of ≤60 mL/min, such as ≤30 mL/min.

Initiation of a subject on sotalol typically refers to the subject who is naïve to sotalol or who has not received sotalol for at least five (5) half-lives of sotalol.

Escalation means increasing the sotalol dosage of a patient already receiving sotalol, for example where a subject is currently taking a specific amount of an oral dose and escalation involves administering one or more IV loading doses to escalate the subject to a higher target oral dose. In embodiments, the IV loading dose is initiated at a time when the next oral dose would have been due.

Sotalol and sotalol hydrochloride (used interchangeably herein) refer to d,l-sotalol hydrochloride.

The terms “treat,” “treating,” and “treatment” refer to any indicia of success in the treatment or amelioration of an injury, disease, or condition, including any objective or subjective parameter such as abatement; remission; diminishing of symptoms or making the injury disease, or condition more tolerable to the subject; slowing in the rate of degeneration or decline; or improving a subject's physical or mental well-being. The treatment or amelioration of symptoms can be based on objective or subjective parameters, including the results of a physical examination, neuropsychiatric examinations, or psychiatric evaluation.

Cₘₐₓ ss is the maximal concentration obtained at steady state.

QT is the interval measured from the start of the Q wave or QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by mathematical correlation of the QT interval and the heart rate.

ΔQTc is the difference between a QTc measurement taken prior to the start of treatment and a QTc measured after the start of treatment (e.g., during loading or maintenance).

The term “about” used herein in the context of quantitative measurements means the indicated amount ±10%. For example, “about 2 mg” can mean 1.8-2.2 mg.

Hospital refers to a medical facility staffed and equipped to provide continuous ECG monitoring and cardiac resuscitation to patients, if needed. Typically, the medical personnel are trained in the management of serious ventricular arrhythmias.

Reducing or shortening the length of a hospital stay refers to reducing/shortening the length of time a patient is admitted for oral sotalol initiation or escalation, for example at a hospital or other facility capable of providing cardiac resuscitation and continuous electrocardiographic monitoring. For example, a patient would typically require a 3-day (72 hour) stay in such hospital or facility to be initiated/escalated on oral sotalol.

Sotalol is indicated for the maintenance of normal sinus rhythm (delay in time to recurrence of atrial fibrillation/atrial flutter (AFIB/AFL)) in patients with symptomatic AFIB/AFL who are currently in sinus rhythm. Sotalol is also indicated for the treatment of life-threatening ventricular tachycardia.

Intravenous sotalol, when used as a loading dose, achieves steady state concentration faster compared to the conventional oral dosing (e.g., 1-3 days for a non-renally impaired patient).

Typically, IV sotalol is diluted for infusion. For example, IV sotalol can be diluted in saline, 5% dextrose in water (D5W), or Ringer's lactate. The dilution volume chosen is one that is convenient for administration and consistent with fluid restriction. A volumetric infusion pump can be used to administer the IV sotalol.

Typically, other antiarrhythmic therapy is withdrawn prior to starting sotalol.

IV and Oral Dosing

In embodiments, the IV loading dose is delivered by way of an IV bolus or infusion over a period of about 1-5 hours, such as for 1 hour, 1.5 hours, 2 hours, 2.5 hours, 3 hours, 3.5 hours, 4 hours, 4.5 hours, or 5 hours, and so on, or within any range with any of these values as lower and upper values. In embodiments, the infusion time and/or the amount of the IV loading dose is selected based on the creatinine clearance of the subject/patient.

In embodiments, the IV maintenance dose is administered to the subject by way of an infusion given for a period of time of 5 hours. The IV maintenance dose in some embodiments is substituted for one or more oral dose. The IV loading or maintenance dose is administered while the patient/subject is admitted to a hospital or other facility capable of providing cardiac resuscitation and continuous electrocardiographic monitoring. In embodiments, one or more of the IV maintenance doses is administered to the patient/subject after release from a hospital or other facility capable of providing cardiac resuscitation and continuous electrocardiographic monitoring.

In embodiments, the IV loading dose is administered in an amount of about 60 mg, about 75 mg, or about 90 mg, such as about 55 mg to about 65 mg, about 50 mg to about 70 mg, about 70 mg to about 80 mg, about 65 mg to about 85 mg, about 85 mg to about 95 mg, about 80 mg to about 100 mg, or about 45 mg to about 105 mg, or about 110-128 mg, or about 99-141 mg or about 49.5-70.2 mg, or about 60-75 mg, or about 73.8-105.6 mg, or from about 90-125 mg, such as from about 112.5 to 125 mg, or about 63-88.2 mg, or about 55-64 mg, or about 75-82.5 mg, or about 82-96 mg, or about 49.5-70.4 mg, or about 73.8-105.6 mg, of from about 90-105 mg, or about 63-88 mg, or about 70-80 mg, or any range in between. The loading dose can also be expressed in mg/min, for example, as a rate of about 0.825-1.17 mg/min. (49.5-70.2 mg), or from about 1.23-1.76 mg/min. (73.8-105.6 mg), or from about 1.65-2.35 mg/min. (99-140.8 mg), or from about 1.05-1.47 mg/min. (66-88.2 mg).

In embodiments, one or more oral or IV maintenance dose is administered, with a first oral or IV maintenance dose being administered about 1-12 hours after initiation of the IV loading dose, such as about 1 hour to about 6 hours, or about 2-8 hours, after the conclusion of the administration of the IV loading dose, such as about 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.25 hours, 5.5 hours, or 5.75 hours after administration of the IV loading dose. In embodiments, the first oral or IV maintenance dose is administered about 1 hour to about 12 hours after conclusion of the administration of the IV loading dose, such as about 2 hours to about 11 hours, about 3 hours to about 10 hours, about 4 hours to about 9 hours, about 5 hours to about 8 hours, or about 6 hours to about 7 hours. In embodiments, the subject/patient is administered the first oral or IV maintenance dose after being released from the hospital or other facility that was providing the monitoring, but the patient/subject can still be monitored in a similar way, such as by using a portable/wearable ECG monitoring system, or not monitored in such a way. In other embodiments, the subject/patient is administered the first maintenance dose while admitted to the hospital or other facility providing the monitoring. In other embodiments, the subject/patient is administered two or more oral doses while admitted to the hospital or other facility providing the monitoring, such as 2, 3, 4 or more oral doses.

In embodiments, the one or more oral dose is selected from about 80 mg, about 120 mg, or about 160 mg, or about 240 mg. In embodiments, more than one oral dose amount is given to a particular patient (for example, a patient receives one or more oral dose at 120 mg and a subsequent higher oral dose of 160 mg or a subsequent lower oral dose of 80 mg, or any combination thereof).

The IV loading or maintenance dose can be intravenously infused into a central or peripheral vein of the patient. The flow rate of the intravenous dose can be adjusted based on the concentration of the sotalol in the intravenous formulation, the desired duration of administration, and the body weight of the patient to achieve a specific loading dose. Thus, longer administration durations of a specific dose (X μg/kg or mg/kg) will typically have slower flow rates compared to shorter administration durations to achieve the same dose.

In embodiments, the IV loading dose is selected based on a patient's creatinine clearance. For example, in some cases a patient/subject with a lower creatinine clearance may receive a lower IV loading dose than a patient with a higher creatinine clearance, or in some cases a patient/subject with a higher creatinine clearance may receive a lower IV loading dose than a patient with a lower creatinine clearance. In some cases, for example, the amount of sotalol hydrochloride appropriate to administer to a subject with a CrCl of 10-30 mL/min or 30-60 mL/min, can be higher than is appropriate for a subject with a CrCl of >90 mL/min.

In embodiments, one or more of the oral or IV maintenance doses is selected based on a patient's change in QTc (for example, a patient that experiences an increase in QTc of less than 20% after receiving the IV loading dose may receive a higher oral or IV maintenance dose than a patient that experiences an increase in QTc of 20% or greater after receiving the loading dose). In various implementations, the change exceeding baseline QTc can be 5%, 10%, 15%, 20%, or 25% over baseline QTc. The QTc can be measured at any point and/or at regular intervals, such as every 10, 15, 20, 30, 45, or 60 minutes during treatment. In some situations, such as if the patient's heart rate is less than 60 bpm, the uncorrected QT interval can be used. Remote ECG monitoring, such as Holter monitoring or event monitoring, may be indicated for long term monitoring of QT interval or QTc. In embodiments, the maintenance dose is changed after administration of a previous maintenance dose due to a change in the patient's QTc (for example, a patient given an oral dose of 120 mg experiences an increase in QTc of 20% or greater, and the next oral dose can be lowered to 80 mg).

In embodiments, the subject/patient is administered the IV loading dose, a first oral or IV maintenance dose, a second oral or IV maintenance dose, and optionally additional oral or IV maintenance doses prior to being released from the hospital or other facility capable of providing cardiac resuscitation and continuous electrocardiographic monitoring. In other embodiments, the IV loading dose and a first maintenance dose are administered in a hospital or other facility that can provide cardiac resuscitation and continuous electrocardiographic monitoring and one or more second or subsequent maintenance doses are administered, such as by the patient according to a prescription, after discharge from the facility/hospital. In embodiments, the IV loading dose is administered in a hospital or other facility that can provide cardiac resuscitation and continuous electrocardiographic monitoring and a first maintenance dose and/or second/subsequent maintenance doses are administered by the patient/subject after discharge from the facility/hospital, but may still be under monitoring, such as self-monitoring.

In embodiments, subsequent IV or oral maintenance dose(s) are given after the first maintenance dose. In embodiments, the subsequent IV or oral maintenance dose(s) are administered at intervals of about 12 hours, about 24 hours, or about 48 hours.

In embodiments, the IV loading dose and/or maintenance dose(s) are given in amounts and at time intervals to enable maximum serum concentration of the subject within a range of ±25% of a Cₘₐₓ at steady state predicted for the subject for an oral dosing protocol of 80 mg, 120 mg or 160 mg, or 240 mg sotalol. In embodiments, the dosing protocol is selected to render the subject/patient capable of achieving Cₘₐₓ ss within 24 hours, such as within 1 hour, of the start of the administration of the IV loading dose. In embodiments, Cₘₐₓ ss is achieved before, during and/or after administration of the first and/or subsequent IV or oral maintenance dose(s). In embodiments, as a result of the IV loading dose and/or the first and/or subsequent IV or oral maintenance dose(s), such as before the first IV or oral maintenance dose, the patient experiences or is capable of experiencing a Cₘₐₓ at steady state that is at least about 85% of the Cₘₐₓ ss, such as about 87%, 90%, 92%, 95%, 97% or 99% of the Cₘₐₓ ss for the patient's dosing protocol.

Indications

In embodiments, sotalol hydrochloride is administered according to any combination of the above or below IV loading and/or oral or IV maintenance doses and durations to treat or prevent a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVCs), paroxysmal supraventricular tachycardia, supraventricular tachycardia, junctional ectopic tachycardia, junctional tachycardia, heart failure, coronary artery disease, and pulmonary artery hypertension, and the like, and for situations where the patient is unable to take an anti-arrhythmic by mouth (NPO). The specific intravenous loading dose and intravenous maintenance dose rate protocols can be selected based upon one or more of the patient's condition, baseline and in-treatment QT interval or QTc, and the IV loading dose can be based on the patient's creatinine clearance as described herein. Other factors affecting selection of the IV loading and oral or IV maintenance dose include patient body weight. Oral maintenance doses of sotalol hydrochloride can be administered to the patient according to available oral dosages (80 mg, 120 mg, 160 mg or 240 mg), QTc, creatinine clearance, body weight, and other factors. Situations where IV loading and/or maintenance doses are appropriate include those in which the patient cannot take oral administration (NPO), or for gastrointestinal conditions resulting in poor absorption, recovery from GI surgery, and/or intensive care. Sotalol administration according to embodiments of the invention is administered to patients naïve to sotalol treatment and/or to patients currently receiving sotalol therapy and/or to patients having previously received sotalol. In embodiments, the sotalol dose (e.g., the loading and/or maintenance dose, whether IV or oral) is determined by patient characteristics including body weight, sex, and/or creatinine clearance.

In embodiments, the patient receiving sotalol hydrochloride has experienced AF, but is in normal sinus rhythm prior to administration of the sotalol hydrochloride loading dose.

The Examples below illustrate various protocols for administering sotalol.

### Example 1

In an embodiment of the invention, sotalol therapy is initiated or escalated by administering to a subject/patient in need thereof a 1 hour IV loading dose of sotalol hydrochloride, with the dosing amount based on creatinine clearance, and one or more 5-hour IV maintenance dose where the timing of the IV maintenance dose is based on creatinine clearance. Example criteria are shown in Table 1 for the 1-hour loading dose amount based on creatinine clearance and the amount of the corresponding 5-hour IV maintenance doses are shown in Table 2.

TABLE 1
IV Sotalol 1-Hr Loading Dosage

| | IV loading dose (1 h infusion) | | | | Min. delay | |
|---|---|---|---|---|---|---|
| | when the dose is going from . . . | | | | to first | IV Maint. |
| Creatinine | Sotalol Initiation | | Sotalol Escalation | | IV Maint. | Dosing |
| Clearance* | 0 to | 0 to | 80 to | 120 to | Dose | Interval |
| (mL/min) | 80 mg** | 120 mg | 120 mg | 160 mg | (hours) | (hours) |
| >90 | 60 | 90 | 75 | 90 | 4 | 12 |
| 60-90 | 82.5 | 125 | 82.5 | 105 | 4 | 12 |
| 30-60 | 75 | 112.5 | 82.5 | 105 | 6 | 24 |
| 10-30 | 75 | 112.5 | 82.5 | 105 | 12 | 48 |

*Calculated using Cockcroft-Gault formula;
**Recommended starting dose

TABLE 2
Sotalol Hydrochloride 5-Hour IV Maintenance Dosing

| Sotalol IV Protocol | Sotalol IV Maintenance Dose | |
|---|---|---|
| Initiation: 80 mg | 75 | mg |
| Initiation & Escalation: 120 mg | 112.5 | mg |
| Escalation: 160 mg | 150 | mg |

The Cockcroft-Gault formulas for creatinine clearance (CrCl) are:

CrCl (male)=((140−age)×weight in kg)/(serum creatinine×72)

CrCl (female)=CrCl (male)×0.85

The recommended starting dose of 80 mg is the FDA recommended dosage. A physician can elect to start a patient on a higher dose (e.g., 120 mg), if deemed appropriate.

The minimum delay to first IV maintenance dose is the time from the end of the IV loading dose infusion to the first IV maintenance dose. In embodiments, the delay to the first IV maintenance dose is chosen from about 4 hours, 5 hours, 6 hours, 7 hours, 8 hours, 9 hours, 10 hours, 11 hours, or 12 hours.

The IV maintenance dosing interval refers to the time between one or more of the IV maintenance dosages. 12 h is B.I.D. (or BID). 24 h is Q.D. (or QD).

In embodiments, the IV loading dose is delivered via IV infusion over a period of 1 hour and is administered/delivered to the patient/subject while admitted to a hospital or other facility capable of providing cardiac resuscitation and continuous electrocardiographic monitoring.

Table 1 shows that the IV loads for initiation of a target dose of 80 mg are 60 mg (>90 mL/min CrCl), 82.5 mg (60-90 mL/min CrCl), and 75 mg (30-60 mL/min CrCl and 10-30 mL/min CrCl). Additional examples of the IV load for the target dose of 80 mg include 49-90 mg, such as 55-85 mg. Further examples include 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, and 85 mg.

Table 1 shows that the IV loads for initiation of a target dose of 120 mg are 90 mg (>90 mL/min CrCl), 125 mg (60-90 mL/min CrCl), and 112.5 mg (30-60 mL/min CrCl and 10-30 mL/min CrCl). Additional examples of the IV load for the target dose of 120 mg include 75-135 mg, such as 82-128 mg. Further examples include 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, and 135 mg.

Table 1 shows that the IV loads for escalation from 80 to 120 mg are 75 mg (>90 mL/min CrCl), and 82.5 mg (60-90 mL/min CrCl, 30-60 mL/min CrCl, and 10-30 mL/min CrCl). Additional examples of the IV load for escalation to 120 mg include 63-96 mg, such as 65-90 mg. Further examples include 65, 66, 67, 68, 69, 70, 71, 72, 73, 74, 75, 76, 77, 78, 79, 80, 81, 82, 83, 84, 85, 86, 87, 88, 89, and 90 mg.

Table 1 shows that the IV loads for escalation from 120 to 160 mg are 90 mg (>90 mL/min CrCl), and 105 mg (60-90 mL/min CrCl, 30-60 mL/min CrCl, and 10-30 mL/min CrCl). Additional examples of the IV load for escalation to 160 mg include 80-120 mg or 88-140.8 mg. Further examples include 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, and 120 mg.

In embodiments, a patient can receive an IV loading dose capable of increasing the patient's oral dosing from 160 mg to 240 mg or initiation of the patient at a 240 mg oral maintenance dose. For example, the dosing amount of the IV loads for escalation from 160 mg to 240 mg or initiation for a 240 mg IV maintenance dose can be in the range of 105 mg to 260 mg, such as 120-140 mg or 110-165 mg (>90 mL/min CrCl), and 130 mg to 240 mg (60-90 mL/min CrCl, 30-60 mL/min CrCl, and 10-30 mL/min CrCl). Additional examples of the IV load for a target of 240 mg include 125-180 mg, 135-200 mg, or 160-220 mg. Further examples include 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, or 260 mg.

The IV loading parameters are selected to enable maximum serum concentration at steady state of sotalol in the subject/patient within 24 hours, such as within 1 hour, of the start of the IV administering and within a range of ±25% of a Cₘₐₓ at steady state predicted for the subject for a corresponding oral dosing protocol of 80 mg, 120 mg or 160 mg or 240 mg sotalol. Once it is determined that the patient/subject is capable of tolerating the sotalol by way of the IV loading dose, IV dosing begins before or after the patient/subject has been released from the hospital or other facility providing monitoring. In embodiments, a first IV maintenance dose is administered before the patient/subject is released from the hospital or other facility that was providing the monitoring. If released, the patient/subject can still be monitored in a similar way, such as by using a portable/wearable ECG monitoring system.

In embodiments, the time of when IV maintenance dosing begins depends on the CrCl of the patient. IV maintenance dosing for CrCl of >90 mL/min and 60-90 mL/min typically begins 4 h after completion of the IV loading dose infusion (e.g., 5 hours after the start of a 1-hour IV loading dose). IV maintenance dosing for a CrCl of 30-60 mL/min typically begins 6 hours after completion of a 1-hr IV loading dose infusion (e.g., 7 h after the start of infusion). IV maintenance dosing for a CrCl of 10-30 mL/min typically begins 12 h after the 1-hr IV loading dose infusion (e.g., 13 h after the start of a 1 h IV loading dose). Additional examples of when the IV maintenance dosing begins for a CrCl of >90 mL/min include 2-6 h after completion of infusion of the IV loading dose. Further examples include 2, 3, 4, 5, to 6 h. Additional examples of when the IV maintenance dosing begins for a CrCl of 60-90 mL/min include 2-6 h after completion of infusion of the IV loading dose. Further examples include 2, 3, 4, 5, to 6 h. Additional examples of when the IV maintenance dosing begins for a CrCl of 30-60 mL/min include 4-8 h after completion of infusion of the IV loading dose. Further examples include 4, 5, 6, 7, to 8 h after. Additional examples of when the IV maintenance dosing begins for a CrCl of 10-30 mL/min include 10-14 h after completion of infusion of the 1-hr IV loading dose. Further examples include 10, 11, 12, 13, to 14 h.

### Example 2

In an embodiment of the invention, sotalol therapy is initiated or escalated by administering to a subject/patient in need thereof a 1-5 hour IV loading dose of sotalol hydrochloride (such as an amount of time more than 1 hour but less than 5 hours) and one or more IV or oral maintenance dose. Examples for the 1-5 hour IV loading dose are in Table 3, and examples of the IV and oral maintenance doses are in Table 4. For example, a subject can be initiated on 80-240 mg or escalated from a prior dose to an amount in the range of 120-240 mg. When being initiated for oral or IV maintenance sotalol, the subject is naïve to sotalol or has not received sotalol for at least five (5) half-lives of sotalol.

The minimum delay to the first IV or oral maintenance dose is the time from the end of the IV loading dose infusion to the first IV or oral maintenance dose. In embodiments, the delay to the first IV or oral maintenance dose is chosen from about 4 hours, 5 hours, 6 hours, 7 hours, 8 hours, 9 hours, 10 hours, 11 hours, or 12 hours.

The IV or oral maintenance dosing interval refers to the time between one or more of the IV or oral maintenance dosages. 12 h is B.I.D. (or BID). 24 h is Q.D. (or QD).

In embodiments, the IV loading dose is delivered via IV infusion over a period of 1.5 hours, 2 hours, 2.5 hours, 3 hours, 3.5 hours, 4 hours, 4.5 hour, or 5 hours (or any range within these values as endpoints of the range) and is administered/delivered to the patient/subject while admitted to a hospital or other facility capable of providing cardiac resuscitation and continuous electrocardiographic monitoring.

TABLE 3
Sotalol Hydrochloride 1-5 Hr. IV Loading Dose
IV loading dose (1-5 hour IV)

| | |
|---|---|
| Initiation | Escalation |

| 0 to | 0 to | 80 to | 120 to | 160 to |
|---|---|---|---|---|
| 80 mg** | 120 mg | 120 mg | 160 mg | 240 mg |
| 50-100 | 75-130 | 60-115 | 70-150 | 100-225 |

**Recommended starting dose

TABLE 4
Sotalol Hydrochloride IV Maintenance Dosing
as a Substitute for Oral Dosing

| | | |
|---|---|---|
| | | Sotalol |
| | Sotalol IV | Oral |
| | Maintenance | Maintenance |
| Sotalol IV Protocol | Dose | Dose |

| | | | |
|---|---|---|---|
| Initiation for 80 mg oral dose | 75 | mg | 80 mg |
| (or 75 mg IV maintenance dose) | | | |
| Initiation & Escalation for | 112.5 | mg | 120 mg |
| 120 mg oral dose (or 112.5 mg | | | |
| IV maintenance dose) | | | |
| Escalation for 160 mg oral dose | 150 | mg | 160 mg |
| (or 150 mg IV maintenance dose) | | | |
| Escalation for 240 mg oral dose | 225 | mg | 240 mg |
| (or 225 mg IV maintenance dose) | | | |

Table 3 shows that the IV loads for initiation of a target oral dose of 80 mg (or target IV maintenance dose of 75 mg) are 50-100 mg. Additional examples of the IV load for the target oral maintenance dose of 80 mg (or target IV maintenance dose of 75 mg) include 40-90 mg, such as 55-85 mg. Further examples include 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, and 85 mg.

Table 3 shows that the IV loads for initiation of a target oral maintenance dose of 120 mg (or target IV maintenance dose of 112.5 mg) are 75-130 mg. Additional examples of the IV load for the target oral maintenance dose of 120 mg (or target IV maintenance dose of 112.5 mg) include 75-135 mg, such as 82-128 mg. Further examples include 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, and 135 mg.

Table 3 shows that the IV loads for escalation from 80 to an oral dose of 120 mg (or escalation to a target IV maintenance dose of 112.5 mg) are 60-115 mg. Additional examples of the IV load for escalation to an oral dose of 120 mg (or target IV maintenance dose of 112.5 mg) include 60-100 mg, such as 65-90 mg. Further examples include 65, 66, 67, 68, 69, 70, 71, 72, 73, 74, 75, 76, 77, 78, 79, 80, 81, 82, 83, 84, 85, 86, 87, 88, 89, and 90 mg.

Table 3 shows that the IV loads for escalation from an oral dose of 120 mg to 160 mg (or escalation to an IV maintenance dose of 150 mg) are 70-150 mg (>90 mL/min CrCl), and 105 mg (60-90 mL/min CrCl, 30-60 mL/min CrCl, and 10-30 mL/min CrCl). Additional examples of the IV load for escalation to 160 mg (oral maintenance) or 150 mg (IV maintenance) include 80-120 mg or 88-140.8 mg. Further examples include 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, and 120 mg.

In embodiments, a patient can receive an IV loading dose capable of increasing the patient's oral dosing from 160 mg to 240 mg (or escalation to an IV maintenance dose of 225 mg) or initiation of the patient at an oral dose of 240 mg (or initiation for an IV maintenance dose of 225 mg). For example, the dosing amount of the IV loads for escalation from 160 mg to 240 mg or initiation for a 240 mg IV maintenance dose can be in the range of 105 mg to 260 mg. Additional examples of the IV load for a target of 240 mg include 125-180 mg, 135-200 mg, or 160-220 mg. Further examples include 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, or 260 mg.

The IV loading parameters are selected to enable maximum serum concentration at steady state of sotalol in the subject/patient within 24 hours, such as within 1-5 hours, of the start of the IV administering and within a range of ±25% of a Cₘₐₓ at steady state predicted for the subject for a corresponding oral dosing protocol of 80 mg, 120 mg or 160 mg or 240 mg sotalol (or corresponding IV maintenance dosing of 75 mg, 112.5 mg, 150 mg or 225 mg). Once it is determined that the patient/subject is capable of tolerating the sotalol by way of the IV loading dose, IV or oral maintenance dosing begins before or after the patient/subject has been released from the hospital or other facility providing monitoring. In embodiments, a first IV or oral maintenance dose is administered before the patient/subject is released from the hospital or other facility that was providing the monitoring. If released, the patient/subject can still be monitored in a similar way, such as by using a portable/wearable ECG monitoring system.

In embodiments, the time of when IV or oral maintenance dosing begins depends on the CrCl of the patient. IV or oral maintenance dosing for CrCl of >90 mL/min and 60-90 mL/min typically begins immediately (for 5-hr IV maintenance dose or oral maintenance dose). IV or oral maintenance dosing for CrCl of 30-60 mL/min typically begins 2 h after completion of the 5-hour IV loading dose infusion. IV or oral maintenance dosing for CrCl of 10-30 mL/min typically begins 8 h after completion of the 5-hour IV loading dose infusion. Additional examples of when the IV or oral maintenance dosing begins for a CrCl of >90 mL/min include 1-6 h after completion of infusion. Further examples include 1, 2, 3, 4, 5, to 6 h. Additional examples of when the IV or oral maintenance dosing begins for a CrCl of 60-90 mL/min include 1-6 h after completion of infusion. Further examples include 1, 2, 3, 4, 5, to 6 h. Additional examples of when the IV or oral maintenance dosing begins for a CrCl of 30-60 mL/min include 3-8 h after completion of infusion. Further examples include 3, 4, 5, 6, 7, to 8 h. Additional examples of when the IV or oral maintenance dosing begins for a CrCl of 10-30 mL/min include 9-14 h after completion of infusion. Further examples include 9, 10, 11, 12, 13, to 14 h.

### Example 3

An example sotalol treatment protocol for a patient experiencing AF but who is currently in normal sinus rhythm is described herein. The male patient, age 60, is admitted to initiate treatment in a hospital or other facility capable of providing cardiac resuscitation and continuous electrocardiographic monitoring. For purposes of timing the maintenance dose, the patient's creatinine clearance is determined to be >90 mL/min. The patient is connected to an electrocardiograph and an initial QTc is determined to be less than 450 ms. A physician determines the patient should be initiated on an oral sotalol dosing regimen of 80 mg by mouth and at a 12-hour interval (timing based on the creatinine clearance). Treatment is initiated with an IV loading dose of sotalol hydrochloride in an amount of about 50-100 mg over a period of about 1-5 hours. The patient's QTc interval is measured during the IV loading dose administration, along with heart rate and blood pressure. The patient's QTc interval does not exceed 500 ms during any of these measurements, and the ΔQTc is less than 20%. The patient would or is expected to achieve a Cₘₐₓ at steady state within or soon after administration of the IV sotalol, such as within 1, 2, 3, 4, 5, or 6 hours, or within 7, 8, 9, 10, 11, 12, 13, 14, 15, 16, 17, 18, 19, 20, 21, 22, 23, or 24 hours, which can be tailored to the particular patient by way of the amount of the dose and infusion time.

The IV loading parameters are selected to enable maximum serum concentration at steady state of the subject/patient within 24 hours, such as within 1 hour, of the start of the IV administering and within a range of ±25% of a Cₘₐₓ at steady state predicted for the subject for an oral dosing protocol of 80 mg sotalol.

Once it is determined that the patient/subject is capable of tolerating the sotalol by way of the IV loading dose, the oral dosing begins after the patient/subject has been released from the hospital or other facility that was providing the monitoring, but the patient/subject can still be monitored in a similar way, such as by using a portable/wearable ECG monitoring system. In other embodiments, the oral dosing begins before the patient/subject has been released from the hospital or other facility. A first oral dose of 80 mg can be administered to the subject about 5-6 hours after initiation of the IV loading dose (or immediately or up to 1 hour after completion of the IV loading dose). The patient's QTc interval can be monitored following administration of the first oral dose. The patient's QTc interval still does not exceed 500 ms and the patient's ΔQTc is less than 20%. The patient would achieve a Cₘₐₓ at steady state within 24 hours of the start of the administration of the IV sotalol, such as within about 1-6 hours or sooner. Subsequent oral doses of 80 mg would be administered by the patient every 12 hours preferably under and using a portable/wearable ECG monitoring system or other monitoring system that can be used at home and/or away from the hospital/facility.

### Example 4

A patient with ventricular arrhythmia is admitted to a hospital (or any facility capable of providing cardiac resuscitation and continuous electrocardiographic monitoring). The male patient, age 60 and naïve to sotalol, is admitted to initiate treatment in a hospital or other facility capable of providing cardiac resuscitation and continuous electrocardiographic monitoring. For purposes of timing the oral maintenance dose, the patient's creatinine clearance is determined to be 60-90 mL/min. The patient is connected to an electrocardiograph and an initial QTc is determined to be less than 450 ms. A physician determines the patient should be initiated on an oral sotalol dosing regimen of 120 mg by mouth at a 12-hour interval (timing based on creatinine clearance). Treatment is initiated with an IV loading dose of sotalol hydrochloride in an amount of about 75-130 mg over a period of about 1-5 hours. The patient's QTc interval is measured during the IV loading dose administration, along with heart rate and blood pressure. The patient's QTc interval does not exceed 500 ms during any of these measurements, and the ΔQTc is less than 20%. The patient would or is expected to achieve a Cₘₐₓ at steady state within or soon after administration of the IV sotalol, such as within 6 hours or within 24 hours of the start of the IV loading dose.

The IV loading parameters are selected to enable maximum serum concentration at steady state of the subject/patient within 24 hours, such as within 1 hour, of the start of the IV administering and within a range of ±25% of a Cₘₐₓ at steady state predicted for the subject for an oral dosing protocol of 120 mg sotalol.

Once it is determined that the patient/subject is capable of tolerating the sotalol by way of the IV loading dose, the oral dosing begins after the patient/subject has been released from the hospital or other facility that was providing the monitoring, but the patient/subject can still be monitored in a similar way, such as by using a portable/wearable ECG monitoring system. In other embodiments, the oral dosing begins before the patient/subject has been released from the hospital or other facility. A first oral dose of 120 mg can be administered to the subject within 9 hours after initiation of the IV loading dose (or immediately or up to 4 hours after completion of the IV loading dose). The patient's QTc interval can be monitored following administration of the first oral dose. The patient's QTc interval still does not exceed 500 ms and the patient's ΔQTc is less than 20%. The patient would achieve a Cₘₐₓ at steady state within 24 hours of administration of the IV sotalol, such as within about 1-6 hours or sooner. Subsequent oral doses of 120 mg would be administered by the patient every 12 hours preferably under and using a portable/wearable ECG monitoring system or other monitoring system that can be used at home and/or away from the hospital/facility.

### Example 5

An example anti-arrhythmic/dofetilide treatment protocol is described herein for a patient with a cardiovascular condition. The female patient previously taking 80 mg sotalol, is admitted to initiate treatment in a hospital or other facility capable of providing cardiac resuscitation and continuous electrocardiographic monitoring. For purposes of timing the IV maintenance doses, the patient's creatinine clearance is determined to be 10-30 mL/min. The patient is connected to an electrocardiograph and an initial QTc is determined to be less than 450 ms. A physician determines the patient should be initiated on an oral sotalol dosing regimen of 120 mg by mouth at a 24-hour interval (timing based on creatinine clearance). Treatment is initiated with an IV loading dose of sotalol hydrochloride in an amount of about 60-115 mg over a period of about 1-5 hours. The patient's QTc interval is measured during the IV loading dose administration, along with heart rate and blood pressure. The patient's QTc interval does not exceed 500 ms during any of these measurements, and the ΔQTc is less than 20%. The patient would or is expected to achieve a Cₘₐₓ at steady state within or soon after administration of the 1-5 hour IV loading dose, such as within 6 hours or 24 hours of the start of the IV loading dose.

The IV loading parameters are selected to enable maximum serum concentration at steady state of the subject/patient within 24 hours, such as within 1 hour, of the start of the IV administering and within a range of ±25% of a Cₘₐₓ at steady state predicted for the subject for an oral dosing protocol of 120 mg sotalol.

Once it is determined that the patient/subject is capable of tolerating the sotalol by way of the IV loading dose, oral dosing begins after the patient/subject has been released from the hospital or other facility that was providing the monitoring, but the patient/subject can still be monitored in a similar way, such as by using a portable/wearable ECG monitoring system. In other embodiments, the oral dosing begins before the patient/subject has been released. A first oral dose of 120 mg can be administered to the subject within 17 hours after initiation of the IV loading dose (or immediately, or up to 12 hours after completion of a 5-hr IV loading dose). The patient's QTc interval can be monitored following administration of the first oral dose. The patient's QTc interval still does not exceed 500 ms and the patient's ΔQTc is less than 20%. The patient would achieve a Cₘₐₓ at steady state within 24 hours of administration of the IV sotalol, such as within about 1-6 hours or sooner. Subsequent oral doses of 120 mg would be administered by the patient every 24 hours preferably under and using a portable/wearable ECG monitoring system or other monitoring system that can be used at home and/or away from the hospital/facility.

### Example 6

An example anti-arrhythmic/dofetilide treatment protocol is described herein for a patient with a cardiovascular condition. The male patient, age 60 and previously taking 120 mg sotalol, is admitted for treatment in a hospital or other facility capable of providing cardiac resuscitation and continuous electrocardiographic monitoring. For purposes of timing the maintenance doses, the patient's creatinine clearance is determined to be 30-60 mL/min. The patient is connected to an electrocardiograph and an initial QTc is determined to be less than 450 ms. A physician determines the patient should be escalated to an oral sotalol dosing regimen of 160 mg by mouth at a 48-hour interval (timing based on creatinine clearance). Treatment is started with an IV loading dose of sotalol hydrochloride in an amount of about 70-150 mg over a period of about 1-5 hours. The patient's QTc interval is measured during the IV loading dose administration, along with heart rate and blood pressure. The patient's QTc interval does not exceed 500 ms during any of these measurements, and the ΔQTc is less than 20%. The patient would or is expected to achieve a Cₘₐₓ at steady state within or soon after administration of the IV loading dose of sotalol, such as within 6 hours or 24 hours of the start of the IV loading dose.

The IV loading parameters are selected to enable maximum serum concentration at steady state of the subject/patient within 24 hours, such as within 6 hours, of the start of the IV administering and within a range of ±25% of a Cₘₐₓ at steady state predicted for the subject for an oral dosing protocol of 160 mg sotalol.

Once it is determined that the patient/subject is capable of tolerating the sotalol by way of the IV loading dose, the oral dosing begins after the patient/subject has been released from the hospital or other facility that was providing the monitoring, but the patient/subject can still be monitored in a similar way, such as by using a portable/wearable ECG monitoring system. In other embodiments, the oral dosing begins before the patient/subject has been released from the hospital or other facility. A first oral dose of 160 mg can be administered to the subject within 11 hours after initiation of the IV loading dose (or immediately or up to 12 hours after completion of the IV loading dose). The patient's QTc interval can be monitored following administration of the first oral dose. The patient's QTc interval still does not exceed 500 ms and the patient's ΔQTc is less than 20%. The patient would achieve a Cₘₐₓ at steady state within 24 hours of administration of the IV sotalol, such as within about 1-6 hours or sooner. Subsequent oral doses of 120 mg would be administered by the patient every 24 hours preferably under and using a portable/wearable ECG monitoring system or other monitoring system that can be used at home and/or away from the hospital/facility.

In embodiments, the patient can be further escalated to a 240 mg oral maintenance dose by administering a subsequent IV dose in the range of 100-225 mg.

### Example 7

An example sotalol IV treatment protocol for a patient receiving an established sotalol oral dosing protocol for arterial fibrillation or arterial flutter (who is in normal sinus rhythm) is described herein. The male patient, age 50, is admitted to a hospital for surgery, such as cardiothoracic surgery. The patient is currently prescribed sotalol hydrochloride for atrial fibrillation in an amount of 80 mg BID. In anticipation of surgery, the patient's status is NPO so the patient is transitioned to IV dosing instead. The patient's creatinine clearance is determined to be >90 mL/min. The patient is connected to an electrocardiograph and an initial QTc is determined to be less than 500 ms. A 1-hour IV loading dosage of sotalol hydrochloride is administered in an amount of 60 mg about 12 hours after the most recent oral dose (see Table 1 for other dosing protocols for a 1-hr IV load with the amount of the dose based on creatinine clearance). Then the patient is given 5-hr IV maintenance doses of sotalol (75 mg) every 12 hours thereafter (from the start of the IV load), with the timing of the IV maintenance doses optionally based on creatinine clearance of the patient. The first IV maintenance dosage is administered over a period of about 5 hours. Additional 5-hr IV maintenance doses are administered every 12 hours from the start of the IV infusion of the just previous IV maintenance dose.

The present disclosure has described particular implementations having various features. In light of the disclosure provided herein, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A method of administering sotalol, comprising:
administering one or more IV dosage of sotalol hydrochloride to a subject, over a period of more than 1 hour and up to 5 hours;
wherein one or more of the IV dosages comprises an IV loading dose and one or more IV or oral maintenance dose;
wherein the IV loading dose, the IV maintenance dose, and the oral maintenance dose comprises an amount of sotalol hydrochloride in the range of 20-260 mg sotalol.

2. The method of claim 1, wherein:
the subject is being initiated on an 80 mg oral maintenance dose and the subject is administered 50-100 mg as the IV loading dose.

3. The method of claim 1, wherein:
the subject is being initiated on a 120 mg oral maintenance dose and the subject is administered 75-130 mg as an IV loading dose.

4. The method of claim 1, wherein:
the subject is being escalated from an 80 mg oral maintenance dose to a 120 mg oral maintenance dose; and
the subject is administered 60-115 mg as an IV loading dose.

5. The method of claim 1, wherein:
the subject is being escalated from a 120 mg oral maintenance dose to a 160 mg oral maintenance dose; and
the subject is administered 70-150 mg as an IV loading dose.

6. The method of claim 1, wherein:
the subject is being escalated from a 160 mg oral maintenance dose to a 240 mg oral maintenance dose; and
the subject is administered 100-225 mg as an IV loading dose.

7. The method of claim 1, wherein the subject has a cardiovascular condition selected from atrial fibrillation, atrial flutter, atrial tachycardia, ventricular tachycardia, hemodynamically stable or unstable ventricular tachycardia, paroxysmal atrial fibrillation, ventricular fibrillation, ventricular arrhythmia, premature ventricular contractions (PVC s), paroxysmal supraventricular tachycardia, supraventricular tachycardia, junctional ectopic tachycardia, junctional tachycardia, heart failure, coronary artery disease, and pulmonary artery hypertension.

8. The method of claim 1, wherein the subject is naïve to sotalol or has not received sotalol for at least five (5) half-lives of sotalol.

9. The method of claim 1, wherein the IV loading dosage is a 5-hour infusion.

10. The method of claim 1, wherein:
the IV loading dose is administered to the subject while admitted to a facility capable of providing cardiac resuscitation and continuous electrographic monitoring.

11. The method of claim 1, further comprising:
administering one or more oral maintenance dose to the subject;
wherein the IV loading dose is administered to the subject while admitted to a facility capable of providing cardiac resuscitation and continuous electrographic monitoring;
wherein the subject is administered one or more of the oral maintenance doses before the subject is released from the facility.

12. The method of claim 1, further comprising:
administering one or more oral maintenance dose to the subject;
wherein the IV loading dose is administered to the subject while admitted to a facility capable of providing cardiac resuscitation and continuous electrographic monitoring;
wherein the subject is administered one or more of the oral maintenance doses after the subject is released from the facility.

13. The method of claim 1, further comprising:
administering one or more oral maintenance dose to the subject;
wherein the IV loading dose and a first oral maintenance dose are administered to the subject while admitted to a facility capable of providing cardiac resuscitation and continuous electrographic monitoring;
the subject is released from the facility capable of providing cardiac resuscitation and continuous electrographic monitoring; and
the subject is administered one or more additional oral dosage of sotalol hydrochloride after release from the facility capable of providing cardiac resuscitation and continuous electrographic monitoring.

14. The method of claim 1, wherein the subject is being treated for atrial fibrillation and/or atrial flutter.

15. The method of claim 14, wherein the subject is in sinus rhythm before being administered the IV loading dose.

16. The method of claim 1, wherein the subject is being treated for ventricular arrhythmia or ventricular tachycardia.

17. The method of claim 1, further comprising:
determining a QT or QTc interval of the subject before administering IV loading dosage and determining a second QT or QTc interval of the subject after administering the IV loading dosage, but before administering one or more oral dosage to the subject;
determining that the second QT or QTc interval is within a selected range;
administering one or more oral maintenance dose to the subject.

18. The method of claim 1, wherein the amount of sotalol hydrochloride in the IV loading dose is more than the amount of sotalol hydrochloride in one or more of the IV or oral maintenance doses.
